(19) United States
(12) Patent Application Publication
(10) Pub. No.: US 2009/0177151 A1
(43) Pub. Date: Jul. 9, 2009

(54) SURGICAL NEEDLE AND METHOD OF GUIDING A SURGICAL NEEDLE

(75) Inventor: Kai Eck, Aachen (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/299,122

(22) PCT Filed: Apr. 26, 2007
(86) PCT No.: PCT/IB07/51555

§ 371 Date: Oct. 31, 2008

(30) Foreign Application Priority Data
May 3, 2006 (EP) 06300436.0

(51) Int. Cl. A61M 5/31

(52) U.S. Cl. 604/95.01

(57) ABSTRACT

A surgical assembly comprising a needle tip (100) connected to a flexible tube (102) filled with a magnetorheo logical (MR) fluid. In order to guide the assembly through body tissue along a desired arbitrary path, the physician successively bends the needle tip (100) to re-orient it toward a desired direction of travel, applies a magnetic field in relation to the MR fluid to cause it to stiffen, applies forward pressure to the device to move it in the desired direction and then removes the magnetic field to return the MR fluid to its initial state.

This invention relates to a surgical needle for penetrating and following a path through body tissue, for use in medical interventions such as surgical procedures or drug-delivery applications, and a method of guiding such a surgical needle along a required path through said body tissue.

There are many known medical interventions, such as surgical procedures and drug-delivery applications, in which a physician is required to guide a surgical device in the form of a needle and catheter along a path through an area of body tissue to a target site.

Currently, a substantially rigid, straight or curved needle has been used for this purpose, wherein the needle body follows the needle tip as the physician applies forward pressure thereto to push the needle through the body tissue.

However, using known techniques and equipment, it is often difficult to achieve precise placement of the device because of the location of the target site in the body, or the proximity of the target site to easily damaged, critical body organs, nerves or other components. It is therefore desirable to provide a surgical device of this type that can be guided precisely along an abitrary, meandering path.

US Patent Application Publication No. US2003/0065373 A1 describes a medical device in the form of a lead to be implanted in or around the heart. The implant is provided with a strip or coating of electroactive rheological material which is caused to stiffen when an electric current is applied to it. Once the implant has been guided through a vein or artery and located at the target site in or around the heart, and electric current is applied to the electroactive Theological material to stiffen the implant and prevent it from being moved from the target as a result of pressure from surrounding tissue. However, such an arrangement is not suitably for precisely guiding a medical device of this type through body tissue, which constantly applies a counteractive force that acts to prevent the accurate guidance of the device.

It is therefore an object of the present invention to provide a surgical device of the above-mentioned type, which can be accurately guided through body tissue along an arbitrary meandering path to enable precise placement of the device, even if the target site is relatively inaccessible or the path or target site is proximal to easily damaged areas of the body. Thus, in accordance with the present invention, there is provided a surgical device for penetrating body tissue and following a path therethrough, said device comprising a resiliently flexible needle tip to which a flexible tube is connected, said flexible tube containing a magnetorheological material, the device further comprising means for selectively applying a magnetic field in relation to said magnetorheological material so as to stiffen said material in said tube and provide a counteractive force against surrounding body tissue.

The provision of a resiliently flexible needle tip, enables the tip to be agitated so as to orient it in the direction in which the device is required to travel, following which, a magnetic field can be applied to the magnetorheological material to stiffen it and the physician can then apply forward pressure to the device to move it a desired amount in that direction. Then, the magnetic field may be removed or at least reduced, the needle re-oriented toward another direction and the process repeated. This can be done as many times as required, thereby enabling an arbitrary meandering path to be followed precisely.

Also in accordance with the present invention, there is provided a method of guiding a surgical device as defined above along a path through body tissue, the method comprising:
a) penetrating said body tissue at a desired location with said needle tip and applying pressure thereto such that the device at least partially enters said body tissue;
b) bending said needle tip so as to re-orient it in a direction in which said device is required to travel;
c) applying a magnetic field in relation to said magnetorheological material so as to cause said material to stiffen in said tube;
d) applying pressure to said device so as to cause said needle tip to move a desired distance through said body tissue in the direction in which said needle tip is oriented; and
e) subsequently at least reducing said magnetic field so as to return said magnetorheological material to its initial state.

Magnetorheological (MR) materials are fluids that solidify into a pasty consistency in the presence of a magnetic field (as molecules assemble in somewhat stiff chains along field lines), and then re-liquify when the force is removed. In its simplest form, an MR fluid can be iron filings suspended in corn oil, but more advanced materials are made from high-tech ceramics mixed with specialised synthetic polymer media. Preferably, the tube is substantially completely filled with MR material, so as to prevent areas where no fluid connection exists.

Steps b), c), d) and e) are beneficially repeated in this order continuously and in rapid succession during a penetration process. The method may further comprise the step of applying pressure to the device when the magnetorheological material has been returned to its initial state so as to cause continued movement of the needle tip through the body tissue in the same direction, as required, until it is required to change the direction of movement. The magnetic fluid may be applied in relation to the MR fluid by means of, for example, external (possibly switchable) electromagnets or by including a magnetic flux with a magnetic field at the extra-corporeal part of the catheter.

These and other aspects of the present invention will be apparent from, and elucidated with reference to, the embodiments described herein.

Embodiments of the present invention will now be described by way of examples only and with reference to the accompanying drawings, in which:

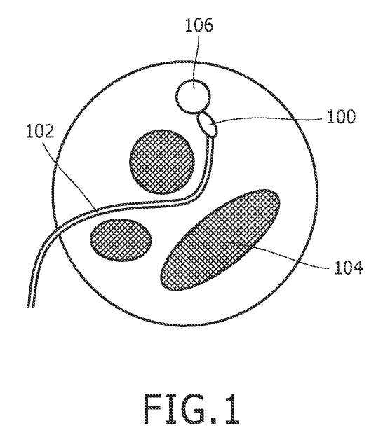

FIG. 1 is a schematic diagram illustrating the manner in which a catheter and needle assembly according to an exemplary embodiment of the present invention can follow an arbitrary path, thereby enabling precise placement of the device at a target site that is relatively difficult to reach; and

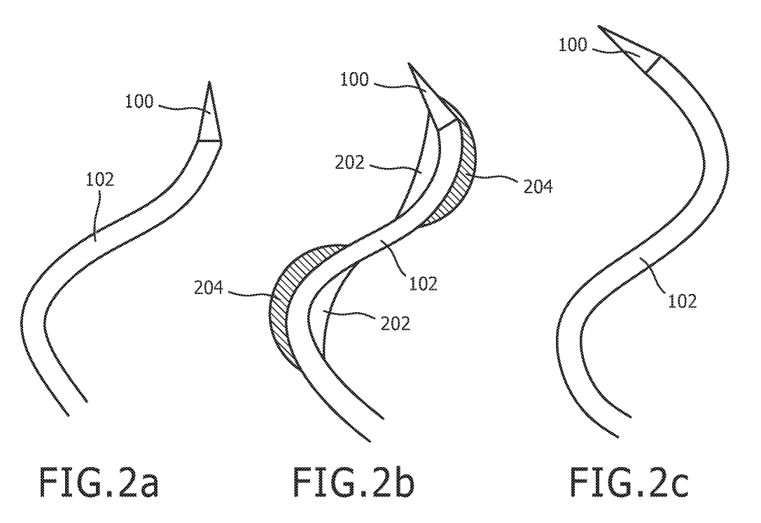

FIGS. 2a, 2b and 2c are schematic diagrams illustrating the principle steps of a method of guiding the assembly of FIG. 1 through body tissue.

Referring to FIG. 1 of the drawings, a surgical device according to an exemplary embodiment of the present invention comprises a bendable needle tip 100 connected to a flexible tube or hose 102 filled with a magnetorheological material. Typical magnetorheological (MR) fluid consists of three parts:
carbonyl iron particles: 20 to 40% of the fluid is made up of these soft iron particles that are just 3 to 5 micrometers in diameter.
a carrier liquid: the iron particles are suspended in a liquid, usually hydrocarbon oil;
additives: to inhibit gravitational settling of the iron particles, promote particle suspension, enhance lubricity, modify viscosity and inhibit wear.

When a magnetic field is applied to the liquid, the carbonyl iron particles line up to make the fluid stiffen into a solid. This is caused by the dc magnetic field making the particles lock into a uniform polarity. How hard the substance becomes depends on the strength of the magnetic field. Once the magnetic field is removed, the particles unlock substantially immediately.

Thus, the MR fluid in the flexible tube 102 can be selectively switched from liquid to stiff and back again by the respective selective application and removal of an external magnetic field. The needle tip 100 is mechanically bendable by a few degrees to allow steering of the device through the body tissue along an arbitrary path, avoiding certain areas 104, to the target site 106.

In the first instance, the needle tip 100 is used to penetrate the subject's body tissue and pressure is applied to the device to cause the it to at least partially enter the body tissue. Referring to FIG. 2a of the drawings, at this stage, the catheter 102 is in some shape (defined by the insertion history) embedded in the body tissue. The needle tip 100 is agitated by the physician so as to re-orient it toward the direction in which it is next required to steer the device. Referring to FIG. 2b, an external magnetic field is applied in relation to the MR fluid in the tube 102, so as the stiffen the fluid, and forward pressure is applied to the device. Such application of pressure leads to positive compression in some areas 204 of the surrounding body tissue and negative stress in other areas 202. The catheter 102 slightly deforms the surrounding tissue as a result and the forward pressure applied at the rear of the device is thus translated to forward movement of the needle tip 100 in the direction of orientation thereof. Referring to FIG. 2c of the drawings, when the external magnetic field is turned down or removed, the catheter 102 follows the pressure exterted thereon by the surrounding tissue and falls back into its path.

Thus, by means of a rapid succession of re-orienting the needle tip 100, solidifying the catheter body 102, pushing the needle tip 100 forward a little and then releasing the stiffness of the catheter body 102 to reduce pressure on the surrounding tissue, the needle tip 100 can be made to follow a desired arbitrary path through the body tissue.

As a result, the present invention permits, for example, targeted delivery of agents in regions where it has previously not been possible, e.g. in stem cell therapy at certain regions of the heart.

It should be noted that the above-mentioned embodiments illustrate rather than limit the invention, and that those skilled in the art will be capable of designing many alternative embodiments without departing from the scope of the invention as defined by the appended claims. In the claims, any reference signs placed in parentheses shall not be construed as limiting the claims. The word “comprising” and “comprises”, and the like, does not exclude the presence of elements or steps other than those listed in any claim or the specification as a whole. The singular reference of an element does not exclude the plural reference of such elements and vice-versa. The invention may be implemented by means of hardware comprising several distinct elements, and by means of a suitably programmed computer. In a device claim enumerating several means, several of these means may be embodied by one and the same item of hardware. The mere fact that certain measures are recited in mutually different dependent claims does not indicate that a combination of these measures cannot be used to advantage.

1. A surgical device for penetrating body tissue and following a path therethrough, said device comprising a resiliently flexible needle tip (100) to which a flexible tube (102) is connected, said flexible tube (102) containing a magnetorheological material, the device further comprising means for selectively applying a magnetic field in relation to said magnetorheological material so as to stiffen said material in said tube (102) and provide a counteractive force against surrounding body tissue.

2. A surgical device according to claim 1, wherein said tube (102) is substantially completely filled with magnetorheological material.

3. A surgical device according to claim 1, comprising means for selectively removing or at least reducing said magnetic field in relation to said magnetorheological material so as to return said material to its original state after said needle tip (100) has travelled a desired distance through said body tissue in a required direction.

4. A surgical device according to claim 3, wherein said means for selectively applying and at least reducing said magnetic field in relation to said magnetorheological material comprise external electromagnets.

5. A surgical device according to claim 4, wherein said electromagnets are switchable.

6. A surgical device according to claim 1, wherein said needle tip (100) is mechanically bendable so as to enable it to be re-oriented in a direction in which said device is required to travel through said body issue.

7. A method of guiding a surgical device according to claim 1 along a path through body tissue, the method comprising:
a) penetrating said body tissue at a desired location with said needle tip (100) and applying pressure thereto such that the device at least partially enters said body tissue;
b) bending said needle tip (100) so as to re-orient it in a direction in which said device is required to travel;
c) applying a magnetic field in relation to said magnetorheological material so as to cause said material to stiffen in said tube (102);
d) applying pressure to said device so as to cause said needle tip (100) to move a desired distance through said body tissue in the direction in which said needle tip (100) is oriented; and
e) subsequently at least reducing said magnetic field so as to return said magnetorheological material to its initial state.

8. A method according to claim 7, wherein steps b), c), d) and e) are repeated in order substantially continually and in rapid succession during a penetration process.

9. A method according to claim 8, further comprising the step of applying pressure to the device when the magnetorheological material has been returned to its initial state so as to cause continued movement of the needle tip (100) through the body tissue in the same direction, as required, until it is required to change the direction of movement.
